(19) United States
(12) Patent Application Publication
(10) Pub. No.: US 2022/0036151 A1
(43) Pub. Date: Feb. 3, 2022

(54) System for Finding Shortest Pathway between Neurons in Neuronal Linkage Pathways

(71) Applicants: National Tsing Hua University, Hsin Chu (TW); National Applied Research Laboratory, Taipei (TW)

(72) Inventors: Ann-Shyn Chiang, Hsin Chu (TW); Ching-Yao Lin, Hsin Chu (TW); Hsiu-Ming Chang, Hsin Chu (TW); Kuen-Long Tsai, Hsin Chu (TW); Chang-Huain Hsieh, Hsin Chu (TW)

(21) Appl. No.: 17/503,794

(22) Filed: Oct. 18, 2021

Related U.S. Application Data
(63) Continuation-in-part of application No. 15/698,211, filed on Sep. 7, 2017.
Application No. 15/698,211 is a continuation-in-part of application No. 13/895,999, filed on May 16, 2013, now abandoned.

(30) Foreign Application Priority Data
Oct. 19, 2012 (TW) 101138827

(51) Int. Cl. G06N 3/04

(52) U.S. Cl. CPC G06N 3/04

(57) ABSTRACT

The present invention discloses a system for finding shortest pathways between neurons in a neuronal network, including a neuronal space generating device for establishing a three-dimensional or higher dimensional neuronal space database (which may be neuron image database), wherein the three dimensional or higher dimensional neuronal space database includes a plurality of neurons distributed therein. A neurons connection determining device is used for determined whether there is a connection between each of the plurality of neurons in the three-dimensional or higher dimensional neuronal space database and the others of the plurality of neurons in the three-dimensional or higher dimensional neuronal space database. A neuronal shortest pathway tables generating device is used for calculating a shortest pathway table of all of a plurality of connected neurons and then stored in a storage device.

### CROSS REFERENCE TO RELATED APPLICATIONS

This application is a Continuation-in-Part of co-pending application Ser. No. 15/698211, filed on Sep. 7, 2017, which is a Continuation-in-Part of application Ser. No. 13/895,999, filed on May 16, 2013, for which priority is claimed under 35 U.S.C. § 120; and this application claims priority of Application No. 101138827 filed in Taiwan on Oct. 19, 2012 under 35 U.S.C. § 119; the entire contents of all of which are hereby incorporated by reference.

### FIELD OF THE INVENTION

The present invention relates to a system for establishing the connecting pathways between neurons within neuronal linkage pathways , and more particularly to a system and a method for finding shortest pathways between neurons through 3D neuron image data.

### BACKGROUND OF THE INVENTION

Researches on brain function may be divided into several levels, from microscopic to macroscopic, such as gene expression, protein biochemistry, cellular morphology, brain neuronal network organizations and animal behavior. Molecular biology, flourished since the 1960s, allows gene manipulation to result in consequences at different scales. In other words, according to basic research in life science, researchers can now make use of technologies to identify genes involved in Drosophila memory and alter them to influence behaviors. Although scientists have a clear understanding of macro-scale biology such as animal behavior and micro-scale biology such as gene expression and perspectives of biology, meso-scale biological research remains under-studied owing to technical limitations including the difficulty to acquire the 3D structure of nerve cells and neuronal network's formation. Now, the integration of biofluorescent labeling and optical section scanning in confocal microscopy gives rise to the possibility of high-resolution digital images of the brain and its neuronal network.

Biologists often can not obtain images (information) of an organism's internal structure without damaging the organism itself. Furthermore, when acquiring biological images, physical limitations of laboratory equipment could only generate a serial of two-dimensional (2D) images instead of three-dimensional images; as a result, the spatial information between organs is not immediately made available. While an invention in 2002, U.S. Pat. No. 6,472,216 presents a sample preparation solution which enables scientists to acquire images from transparent whole mount samples.

Owing to the technological advances in the twentieth century, it is generally accepted that a completely modular brain model can depict its functional validity. Therefore, the interpretation of brain function can be analytically and anatomically described based on the interactions among different brain regions or even neurons. Accordingly, 3D image reconstruction technology can be applied to build models of major compartments of the brain, and at the same time, merge the anatomy of neuropils or neurons with the function of neuronal networks in the brain.

Although the information processing and transmission mechanism of the human brain fascinates scientists and are main research topics, owing to the fact that the human brain has 100 billion neurons, plus human's relatively long-life span and the legal and moral restriction, human genes cannot be manipulated at will. Neuroscientists thus turn their investigation to other organisms, e.g. mice, zebrafish and Drosophila. For instance, the Drosophila brain only has about 135,000 neurons, but can still exhibit complex memory and learning behaviors; consequently, it has become one of the most popular and important research targets in neuroscience. In addition, Drosophila genes have been entirely sequenced, and its short life cycle (approximately 60 days) further makes it a feasible research target. Transparent brain tissues of Drosophila may be prepared by utilizing tissue clearing solution invented by one of the inventors of the present application (U.S. Pat. No. 6,472,216 B1 and TW patent No. 594005), and a system is established to collect single neuron image in Drosophila brain (TW patent No. 1291630 and U.S. Pat. No. 7,742,878 B2). Nowadays, there are nearly twenty thousand brain neurons collected in the Drosophila brain, which are arranged appropriately according to their related physiological locations and therefore can be utilized as the basis for the research of the structure and function of the brain (Current Biology 2011, vol:21: p1-11 Three-dimensional reconstruction of brain-wide wiring networks in Drosophila at single-cell resolution; Pacific Visualization Symposium (PacificVis), 2011 IEEE, 1-4 March: 35-42 The Neuron Navigator: Exploring the information pathway through the neural maze).

For brain function research, the message transmission pathways may facilitate to solve the puzzle of how the brain works. Once the shortest pathway between two neurons can be found, it may provide clues to facilitate simulation of the transmission and interpretation of neural signals.

The existing techniques can obtain high resolution images of neurons and acquire three dimensional stereoscopic models by utilizing three-dimensional stereoscopic image reconstruction techniques. The interconnections among neuropils do not imply linkage neurons are directly connected because there are additional neurons with fibers tangled within each neuropil mediating signal transmissions. Thus, for the shortest pathway between two neurons, there is no method in the past to present the shortest pathway between neurons; especially each neuron possesses irregular shape and complicated terminal branches. Therefore, there is a demand for a new method which can solve the problems and visualize results, otherwise further study on the transmission pathways in the neuronal network is impossible.

### SUMMARY OF THE INVENTION

One object of the present invention is to solve the problem of how to find the shortest pathway between every two neurons from the neuron images in three dimensional or higher dimensional neuronal linkage pathways.

To achieve the aforementioned object, the present invention provides a method for finding shortest pathways between neurons, with irregular shapes and complicated terminal branches, among neuronal linkage pathways. The method includes the following steps: establishing a neuron image database from three dimensional or higher dimensional neuron images by a processing device in a storage device, where the neuron images include a plurality of neurons stored within. Then, whether there is a connection between each of neurons and the other neurons in the three dimensional or higher dimensional neuron image database is determined by the processing device. Subsequently a shortest pathway table of all of the connected neurons is calculated via an All-pairs Shortest Paths algorithm and is temporarily or permanently stored in the storage device. Therefore, as long as any two connected neurons are selected from the three dimensional or higher dimensional neuron image database, the shortest pathway between the two neurons can be obtained in real-time by looking up the aforementioned shortest pathway table in the storage device.

In some embodiments of the present invention, the method described above may be performed by a local processing device. Alternatively, in other some embodiments of the present invention, the method described above may be performed by a processing device in a remote server which is connected to network.

In the aforementioned embodiments, the three dimensional or higher dimensional neuronal space database may be constructed from a plurality of unit voxels in the multi-dimensional neuron image space.

Furthermore, the determining step mentioned above includes establishing a connection matrix (CM) between the plurality of neurons from the three dimensional or higher dimensional neuronal space database, where CM(i, j)=1 denotes that there is a connection between a neuron ni and a neuron nⱼ; CM(i, j)=0 denotes that there is no connection between the neuron ni and the neuron Ilᵢ.

A shortest pathway matrix (represented by SP) and a predecessor matrix (represented by Pred) are generated in the All-pairs Shortest Paths algorithm. When SP(i, j)=∞, it denotes that there is no connection between a neuron ni and a neuron nⱼ; when Pred(i, j)=nil (i.e. empty set), it denotes that a shortest pathway between a node i and a node j is Edge(i, j); when Pred(i, j)=k, it denotes that the shortest pathway from the node i to the node j passes a node k, where the node k is the predecessor of the node j on the shortest pathway from the node i to the node j. The predecessor matrixes may be stored in the form of tables and the shortest pathway between any two nodes is established by looking up the predecessor table. The shortest pathway within all nodes needs not to be calculated repeatedly.

Based on the above description, neurology researchers may establish the database by employing the neuronal images interested via the aforementioned method and can then find out the shortest pathways between every two neurons in the database easily and rapidly through the method described in this invention, so as to facilitate the studies on neuronal functions and disease with persuasive anatomical descriptions.

### BRIEF DESCRIPTION OF THE DRAWINGS

FIG. 1 illustrates a flow chart of the method for finding the shortest pathways between neurons in neuronal linkage pathways in accordance with the present invention;

FIG. 2 illustrates an architecture diagram of the system applied to perform the method for finding the shortest pathways between neurons in neuronal linkage pathways in accordance with one embodiment of the present invention;

FIG. 3 illustrates an architecture diagram of the system applied to perform the method for finding the shortest pathways between neurons in neuronal linkage pathways in accordance with another embodiment of the present invention;

FIG. 4 illustrates a diagram of a unit voxel utilized to construct the three-dimensional neuronal space database in accordance with the present invention;

FIG. 5A illustrates an architecture diagram of the manipulation interface applied in the present invention;

FIG. 5B illustrates a diagram of the manipulation interface applied in the present invention in accordance with one embodiment;

FIG. 6 illustrates an architecture diagram of the system applied to perform the method for finding the shortest pathways between neurons in neuronal linkage pathways in accordance with another embodiment of the present invention;

FIG. 7 illustrates an architecture diagram of the system applied to perform the method for finding the shortest pathways between neurons in neuronal linkage pathways in accordance with yet another embodiment of the present invention; and

FIG. 8 illustrates a functional block diagram of the processing device applied to perform the method for finding the shortest pathways between neurons in neuronal linkage pathways in accordance with one embodiment of the present invention.

### DETAILED DESCRIPTION OF THE PREFERRED EMBODIMENT

The present invention will now be described with the embodiments and aspects taken together with the accompanying drawings and these descriptions interpret structure and procedures of the present invention only for illustrating but not for limiting the Claims of the present invention. As used herein, references to one or more “embodiments” are to be understood as describing a particular feature, structure, or characteristic included in at least one implementation of the invention. Thus, phrases such as “in one embodiment” or “in an alternate embodiment” appearing herein describe various embodiments and implementations of the invention, and do not necessarily all refer to the same embodiment. However, they are also not necessarily mutually exclusive.

The embodiments and details which will be described in detail below include descriptions in connection with the drawings. The description in connection with the drawings may be described in some embodiments or in all embodiments below as other potential embodiments or implementations of the invention concepts presented herein. The embodiments of the present invention are described in great detail below. Please refer to the drawings.

The present invention relates to a method for finding the shortest pathways between neurons in neuronal linkage pathways through neuron image data. In other words, the method for finding the shortest pathways between neurons in neuronal linkage pathways is utilized to receive information of two neurons (for example names or IDs), calculate the connecting neurons between the two neurons and output complete pathway information.

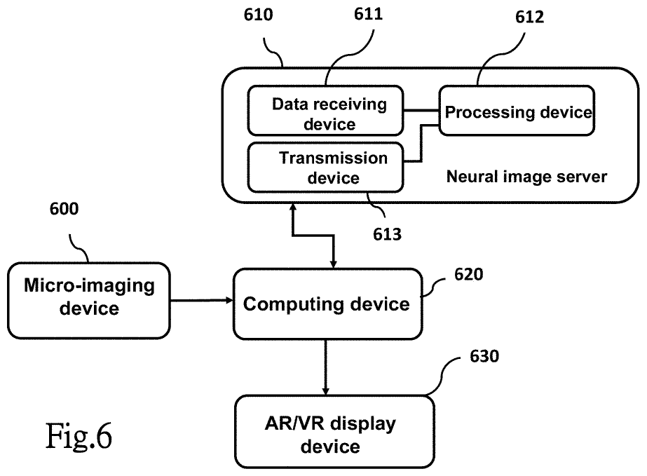

As shown in FIG. 6, it illustrates an architecture diagram of the system applied to perform the method for finding the shortest pathways between neurons in neuronal linkage pathways in accordance with one embodiment of the invention. In this exemplary example, the system includes a micro-imaging device 600, a neuronal image server 610, a computing device 620 and a AR/VR display device 630. The 3D (three dimensional) image is generated by inputting Drosophila neuronal image obtained from the micro-imaging device 600. The 3D image is acquired from a fluorescent-labeled specimen scanned by a laser scanning microscope. During the scanning process, at least part of the sample is scanned by laser. The cross-section of different depths of the sample was scanned in accordance with a predetermined order; the resulting scanned images were numerous plane images at different depths. Images from different slices of the same stack were combined to form a complete image; and then the resulting 3D image consisting of different cross-sections was generated by a computer software.

The neuronal image server 610 includes a data receiving device 611, a processing unit 612 and a transmission device 613. The data receiving device 611 and the transmission device 613 are coupled to the processing unit 612. The transmission device 613 may be a wireless transmission device, such as Wi-Fi device or blue tooth device or a wired transmission device, such as wired network routing device. The micro-imaging device 600 may include a transmission device. The data retrieving device 635 of the computing device 200 is used for retrieving the 3D image data obtained from the micro-imaging device 600 in accordance with the retrieval condition, and followed by outputting the 3D image data to the computing device 620 via a transmission device. The data retrieving device 635 refer to a digital hardware to deliver the 3D image data. The data receiving device 611 of the neuronal image server 610 is used for receiving 3D neuronal space database, shortest pathway tables and predecessor matrix tables from the computing device 600 via a transmission device 613 and 632 in accordance with the receiving condition. It should be noted that the neuronal image server 610 may also include other auxiliary units, or necessary components which are not so relevant to the present invention. However, it should be easily appreciated by the person having ordinary skill in the art that the neuronal image server 610 may or may not include these components, which are therefore omitted herein.

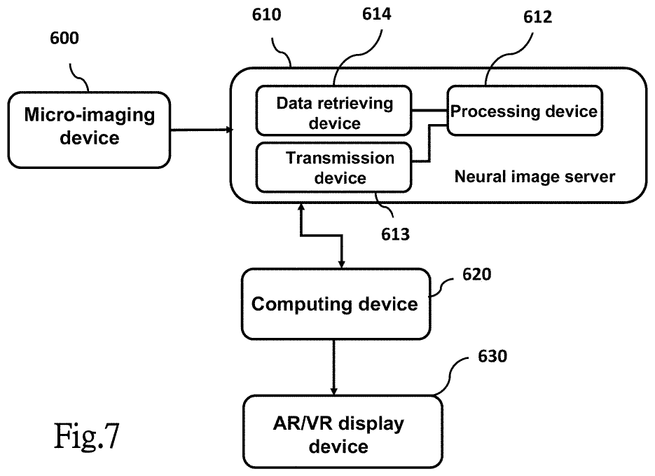

In another embodiment, the data retrieving device 614 of the neuronal image server 610 is used for retrieving the 3D image data obtained from the micro-imaging device 600 in accordance with the retrieval condition, and followed by outputting the 3D image data to the neuronal image server 610 via a transmission device, shown in FIG. 7. The data receiving device 636 of the computing device 620 is used for receiving 3D neuronal space database, or shortest pathway tables and predecessor matrix tables from the neuronal image server 610 via a transmission device 613 and 632 in accordance with the receiving condition.

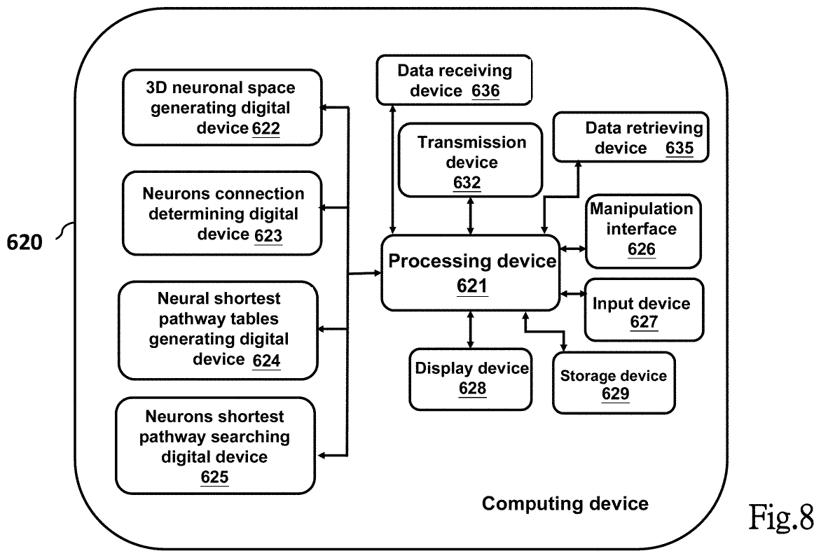

The computing device 620 includes a personal computer, such as a desktop or laptop computer, network server, or other similar device. Examples of a data link between the devices in the system include USB, USB X.0 (X=2, 3 . . . ), Ethernet, WiFi, or WiGig (Wireless Gigabit Alliance). The computing device 620 generally includes a processing device 621, an input device 627 such as keyboard or touch panel, a display device 628 such as touch panel display, a storage device 629 such as memory, hard drive, and a transmission device 632, as shown in FIG. 8. The transmission device 613 is compatible with the transmission device 632 for facilitating data transfer. In this embodiment, the computing device 620 further includes a 3D neuronal space generating digital device 622, a neurons connection determining digital device 623, a neuronal shortest pathway tables generating digital device 624, a neuronal shortest pathway searching digital device 625 and a manipulation interface 626, as shown in FIG. 8. The neuronal space generating digital device 622 for collecting the 3D neuronal image positions, and establishing a three-dimensional or higher dimensional neuronal space database (which may be neuron image database), the three dimensional or higher dimensional neuronal space database includes a plurality of neurons distributed therein. A neurons connection determining digital device 623 is used for keeping connection matrix (CM) and determining whether there is a connection between each of the plurality of neurons in the three-dimensional or higher dimensional neuronal space database and the others of the plurality of neurons in the three-dimensional or higher dimensional neuronal space database. A neuronal shortest pathway tables generating digital device 624 is used for collecting results of connections and calculating a shortest pathway table of all of a plurality of connected neurons and then stored in a storage device. The neuronal shortest pathway searching digital device 625 is employed to calculate all pairs shortest paths (APSP) and comparing results. The above-mentioned devices are electrically coupled to the processing device 621. In one embodiment, the manipulation interface 626 is stored/disposed in the storage device 629. It should be noted that the computing device 620 may further include other auxiliary devices or components which are not so relevant to the present invention. However, it should be easily appreciated by the person having ordinary skill in the art that the computing device 620 may or may not include these auxiliary units or components, which are therefore omitted herein.

The present invention provides the AR/VR (Augmented reality/Virtual reality) display device 630 capable of improving a stereoscopic feeling of the 3D neuronal image.

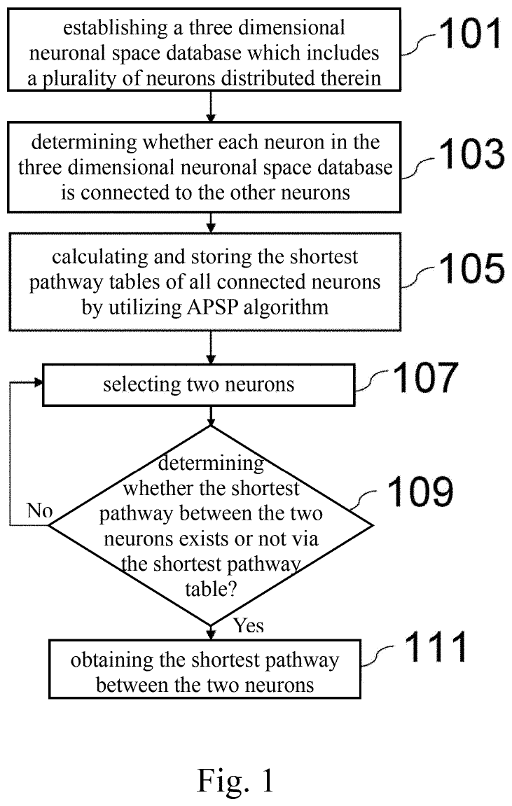

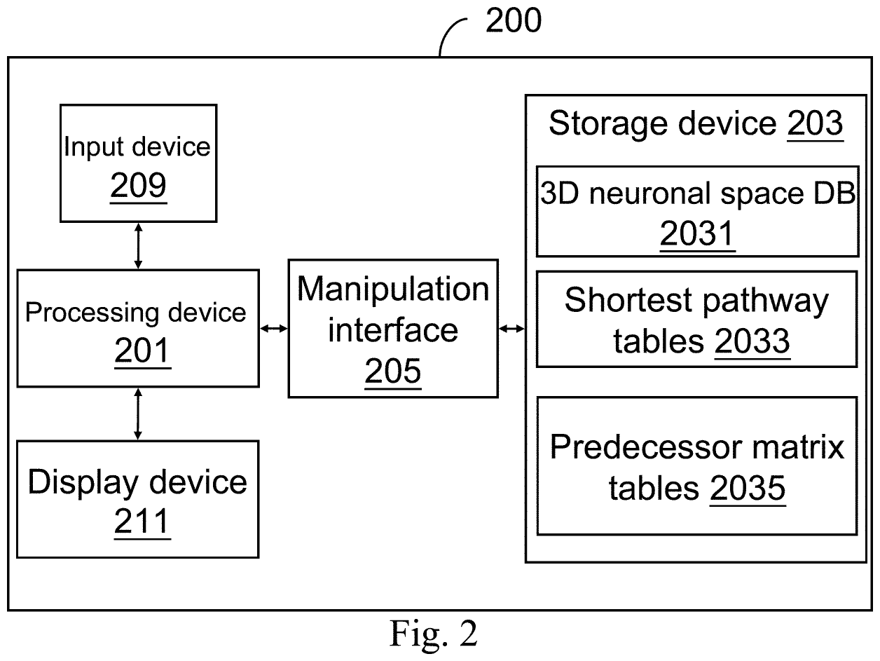

Please refer to FIG. 1, which is a flow chart of the method for finding the shortest pathways between neurons in neuronal linkage pathways, taken together with FIGS. 2 to 5.

Firstly, the first step of the method for finding the shortest pathways between neurons through neuron image data disclosed by the present invention is to establish a three-dimensional neuronal space database (also referred to as three-dimensional neuron image database) which includes a plurality of neurons distributed within three-dimensional neuronal space database (step 101). The three-dimensional neuronal space database is generated by the 3D neuronal space generating digital device 622.

It should be noted that although this embodiment is described with the three-dimensional neuronal space database as an example, the present invention is not limited to the three-dimensional neuronal space database but may be applied to higher dimensional neuronal space databases.

Please refer to FIG. 2 first, which is an architecture diagram of the system applied to perform the method for finding the shortest pathways between neurons in neuronal linkage pathways in accordance with one embodiment. In this example, the system 200 may be performed by a single processing device. The system (processing device) 200 may include a processing device 201 to process the operation of the system (processing device) 200; and a storage device 203 to store data. Furthermore, the system 200 may also include a manipulation interface 205 connected to the processing device 201 and the storage device 203 respectively. The storage device 203 includes storage spaces, and three-dimensional neuronal space databases 2031, shortest pathway table(s) 2033 and predecessor matrix table(s) 2035 (which will be described in detail below) may be stored in the storage spaces. In the present invention, the manipulation interface 205 and the storage spaces of the storage device 203 are connected to each other, such that the user can establish and manipulate the three-dimensional neuronal space database 2031 through the manipulation interface 205. Moreover, in this exemplary example, the system (processing device) 200 further includes an input device 209 and a display device 211, both of which are electrically coupled to the processing device 201, such that the display device 211 may display the pictures of the manipulation interface 205 in order for the user to view them, and the user may manipulate the manipulation interface 205 through the input device 209.

In some embodiments of this exemplary example, the system (processing device) 200 may be composed of a computer system, where the processing device 201 may be performed by a central processing unit (CPU), and the storage device 203 may include any kinds of readable recording medium, such as magnetic storage device, optical storage device or volatile storage device, and shall not be limited thereto. Further, the input device 209 may be performed by any kinds of components or devices for computer input, for instance mouse, keyboard, touch panel, etc, and shall not be limited thereto. The display device 211 may also be performed by any kinds of components or device for display, for example liquid crystal display (LCD) device, etc.

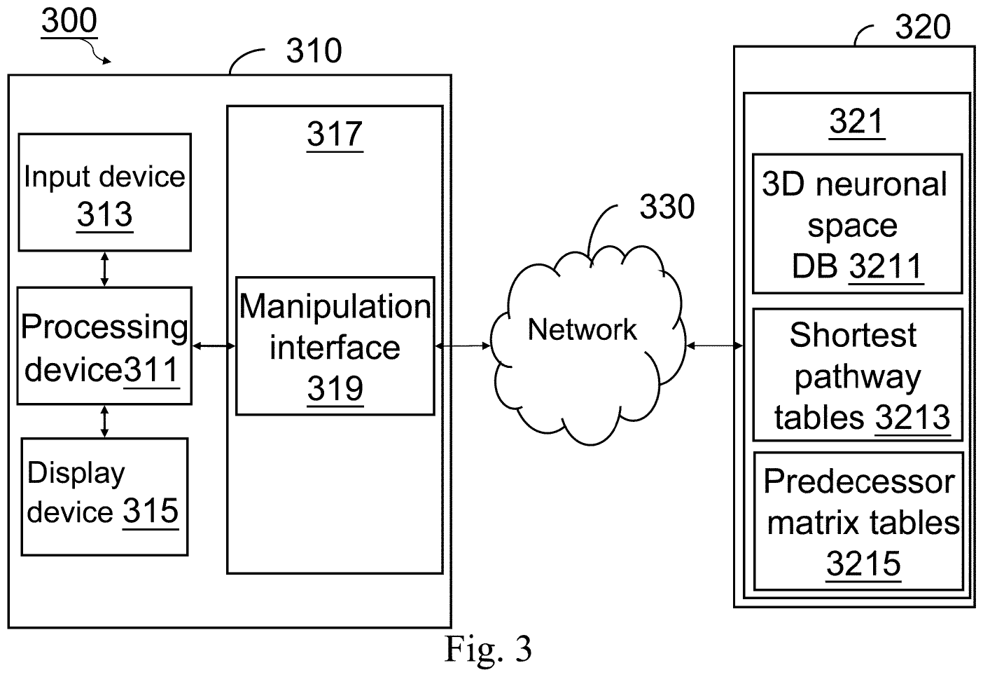

Furthermore, please refer to FIG. 3, which is an architecture diagram of the system applied to perform the method for finding the shortest pathways between neurons in neuronal linkage pathways in accordance with another embodiment. In this exemplary example, the system 300 may be composed of a processing apparatus 310, a server 320 and a network 330. The processing apparatus 310 generally includes a processing device 311, an input device 313, a display device 315 and a storage device 317. The storage device 317 is electrically coupled to the processing device 311, and a manipulation interface 319 is disposed therein. The input device 313 and the display device 315 are electrically coupled to the processing device 311 respectively. It should be noted that the processing device 310 may further include other auxiliary units or components which are not so relevant to the present invention. However, it should be easily appreciated by the person having ordinary skill in the art that the processing device 310 may or may not include these auxiliary units or components, which are therefore omitted herein. In some embodiments of this exemplary example, the processing device 311 may be a central processing unit (CPU), and the storage devices 321, 317 may include any kinds of readable recording medium, such as magnetic storage device, optical storage device, volatile storage device, or hard drive. Further, the input device 313 may be performed by any kinds of components or devices for computer input, for instance mouse, keyboard, touch panel, etc, and shall not be limited thereto. The display device 315 may also be performed by any kinds of components or device for display, for example liquid crystal display (LCD) device, etc.

The server 320 generally includes a storage device 321. A three-dimensional (3D) neuronal space database (DB) 3211 is established and stored in the storage spaces of the storage device 321. Similarly, the storage device 321 also stores shortest pathway tables 3213 and predecessor matrix tables 3215 (which will be described in detail below). The three-dimensional (3D) neuronal space database (DB) 3211 shortest pathway tables 3213 and predecessor matrix tables 3215 are all stored in the storage device 321 which is a computer readable storing media.

It should be noted that the server 320 may also include other auxiliary units, or necessary components which are not so relevant to the present invention. However, it should be easily appreciated by the person having ordinary skill in the art that the server 320 may or may not include these components, which are therefore omitted herein.

In this embodiment, the processing apparatus 310 and the server 320 may connect to each other through a network. More specifically, the user can employ the manipulation interface 319 in the processing device 310 to connect with the three-dimensional neuronal space database 3211 established in the storage device 321 in the remote server 320 through the network 330, and can establish and manipulate the three-dimensional neuronal space database 3211 via the manipulation interface 319.

Similarly, in some embodiments of this exemplary example, the processing device 310, the server 320 and the network 330 may be performed and operated by any kinds of existing processing devices, servers and networks, which are therefore omitted therein.

In the present invention, the establishment of the three-dimensional neuronal space database (which may also be referred to as three-dimensional neuron image database) 2031, 3211 is derived from the concept of spatial database management system (SDBMS). SDBMS is applied to geographic information system (GIS) initially, and is also utilized in different fields later. For instance, molecular biologists can employ SDBMS to search and compare the structures of different genes, and astronomers can also utilize SDBMS to search and analyze the constellations. Therefore, as long as all of the objects to be searched are located in the same coordinate space, SDBMS can be employed to sort and analyze them to help the researchers.

The method for establishing the three-dimensional neuronal space database utilized in the present invention may refer to the disclosure in a Taiwan Patent Application Number 098115595, entitled “A METHOD FOR SEARCHING AND CONSTRUCTING 3D IMAGE DATABASE”.

The method for establishing the three-dimensional neuronal space database (which may also be referred to as three-dimensional neuron image database) 2031, 3211 may be generally divided into the preprocessing of three-dimensional image and management of the neuron image. Each set of Drosophila brain neuronal images may be obtained, and each set of the obtained Drosophila brain neuronal images was warped and registered by employing the preprocessing steps and may be represented with tracing lines. The ends of the tracing lines may be the terminals of the neurons. These terminals represent the portions of the neurons which are responsible for information exchange. Thus, if the distance between the terminals of two neurons is less than a certain range, we assume that these two neurons are connected to each other, and the information exchanges happen between these two neurons.

Each neuronal image datum and each tracing line may be warped into a normalized Drosophila brain coordinate system, and the space occupied by neuronal images and each terminal have corresponding spatial coordinates. Thus, a Spatial Database Management System (SDBMS) may be employed to store and manage the information included in the Drosophila brain images, and the relationship querying function may be provided through the Drosophila neuronal image information stored in SDBMS.

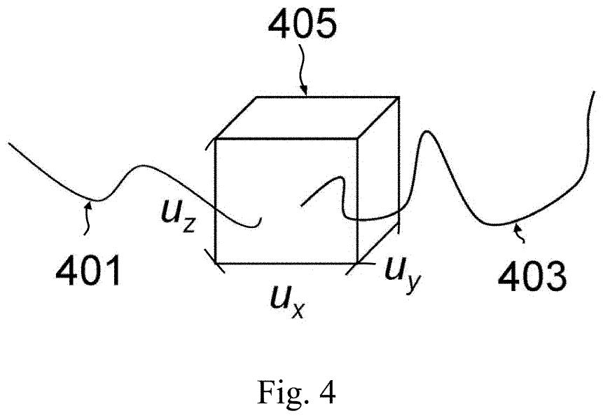

In the present invention, the establishment of the three-dimensional neuronal space database 2031, 3211 is described as follows: a unit voxel u (please refer to FIG. 4) is defined in the space where all of the registered neuronal images are located (it is assumed that the dimensions of neuron image space in X, Y and Z directions are set to be Sx, Sy and Sz respectively), and the size of the unit voxel is set to be ux×uy×uz. The defined unit voxel u may be of different size, and the larger the unit voxel u is, the higher the fault tolerance will be. Dx, Dy and Dx denotes the number of the unit voxels which each dimension in this space can accommodate, i.e. Dx=Sx/ux, Dy=Sy/uy and Dz=Sz/uz. In other words, this neuronal space may include a number of Dx×Dy×Dz unit voxels.

In one embodiment of the present invention, the unit voxel may be 2×2×2 pixels, and the standard brain space may be 1944×1222×740 pixels, but is not limited thereto.

After the three-dimensional neuronal space database 2031, 3211 which includes a plurality of neurons distributed therein is established, it is determined whether each neuron in the three-dimensional neuronal space database 2031, 3211 is connected to the other neurons (step 103) by the neurons connection determining module 623.

In the present invention, when the database 2031, 3211 is established, the connecting relationship between two neurons may be found by querying the database to know that if the terminals of these two neurons are located within the same unit voxel. There may be errors in the processing procedure of the neuronal images. Therefore, a threshold may be defined. In this embodiment, the size of a unit voxel is set as the threshold. Thus, if any one of terminals of one neuron and any one of terminals or part of the trace of another neuron are located within the same unit voxel, it is taken that a connection exists between the two neurons. That is, information is transmitted between the two neurons at such point. With reference to FIG. 4, the cube 405 represents a unit voxel, and the unit voxel 405 in which a terminal of a first neuron 401 is located has a second neuron 403 passing through. Thus, it may be considered that there is a connection between the first neuron 401 and the second neuron 403.

Therefore, when the database 2031, 3211 is established, the database 2031, 3211 may be employed to find the neuron(s) connected with all other possible neurons. For instance, in order to find neuron(s) connected with a neuron ni, the first step is to find out all of the unit voxels through which the neuron ni passes, and then all of the neurons which pass through these unit voxels can be found from the database 2031, 3211.

In other word, for each unit voxel u in a number of Dx×Dy×Dz unit voxels, the neurons passing through the unit voxel u are found, and this related information is stored in the three-dimensional neuronal space database (also referred to as three-dimensional neuron image database) 2031, 3211 established in step 101. The database 2031, 3211 mainly includes the location of the unit voxel (ux, uy, uz), and the content is the names of all of the neurons passing through this unit voxel (which may be represented by ID (identification number)).

Connection matrix (also referred to as adjacency matrix) between neurons may be built up via the aforementioned concepts. It is assumed first herein that there are m records stored in the database 2031, 3211, i.e. there are m neurons. Thus, a m×m matrix, which is represented by CM, may be established. When CM(i, j)=1, it denotes that there is a connection between the neuron ni and the neuron nⱼ. Contrarily, when CM(i, j)=0, it denotes that there is no connection between the neuron ni and the neuron nⱼ. In such way, the values in the whole CM matrix are filled, and this CM matrix is the connection matrix showing the connecting relationship between neurons.

In the present invention, when the connection matrix between every two neurons is built up, the connection matrixes among all neurons may be converted into a undirected graph to represent them. Under this structure, each neuron is equivalent to a node. A connection between two neurons is equivalent to that there is a connecting edge between two nodes. Thus, if there is a connection between the neuron ni and the neuron nⱼ, i.e. CM(i, j)=1, it denotes that there is an edge with a weight of 1 between the node i and the node j. The algorithm of graph analysis in graph theory may then be applied to the neuron connection structure. In one exemplary example of the present invention, the neuronal images to be used are the neuronal images of Drosophila brain, which has about thirteen thousand neurons for now. Therefore, it is equivalent to that the shortest pathways have to be found from a graph with about thirteen thousand nodes. It's better to find out the shortest pathway in real-time. Traditionally, the shortest pathways are usually calculated by utilizing Dijkstra's algorithm, in which a certain node is utilized as a starting node, and the shortest pathways from the starting node to all of the other nodes are then calculated. If the traditional method is applied to the present system, the shortest connecting pathways between one node among ten thousand nodes and the other nodes among the ten thousand nodes have to be searched respectively. It is very difficult for the processing devices 200, 310 to find the shortest pathways in real-time.

In the present invention, all pairs shortest paths (APSP) may be calculated by employing Floyd-Washall Algorithm, which is also referred to as APSP algorithm. This calculating method can calculate the shortest pathways among all neurons which are connected to one another. Although there are thirteen thousand neurons in this embodiment and the computation is very time-consuming, i.e. the dimension of the connection matrix is around 13000×13000, the present invention calculates the shortest pathways among all connected neurons only one once and establishes shortest pathway matrix tables to store the shortest pathways (step 105) by the neuronal shortest pathway tables generating module 624. The APSP algorithm is stored in the neuronal shortest pathway tables generating module 624.

When the APSP algorithm is applied to the computation of the connection matrixes of the present invention, two matrixes will be generated, where one is the shortest path (SP) matrix, i.e. SP(i, j) is the distance of the short pathway from the node i to the node j, and the other is predecessor matrix (Pred). Thus, if SP(i, j)=∞, it denotes that there is no pathway to connect the node i and the node j. There are two situations for Pred(i, j) as follow: when Pred(i, j)=nil (i.e. an empty set), it represents that the shortest pathway between the node i and the node j is Edge(i, j); when Pred(i, j)=k, it represents that the shortest pathway from the node i to the node j may pass the node k, where the node k is the predecessor of the node j on the shortest pathway from the node i to the node j. The predecessor matrixes and the shortest pathway matrixes may be generated by the neuronal shortest pathway tables generating module 624.

Therefore, in order to find the shortest pathway (Path(i, j)) between the neuron IL and the neuron nⱼ, the SP matrix needs to be checked first. If SP(i, j) is not ∞, it denotes that the shortest pathway exists. Then, the shortest pathway may be found from the Pred matrix. Moreover, if Pred(i, j)=k, the shortest pathway between the node i and the node j is Path(i, k) plus Path(k, j), such that the shortest pathway between the neuron ni and the neuron nⱼ can be found.

The predecessor matrixes and the shortest pathway matrixes may be stored in the form of tables as the predecessor matrix table 2035, 3215 and the shortest pathway matrix table 2033, 3213 respectively. When the shortest pathway between any two neurons is to be searched, the pathway may be found directly by look-up-table (LUT) and the APSP algorithm needs not to be performed every time.

Thus, the shortest pathway tables (predecessor tables) may be formed through the aforementioned method (by the neuronal shortest pathway tables generating digital device 624) and may be then stored in the storage device 203, 321.

After the shortest pathway tables are completed, as long as two neurons may be selected from the three-dimensional neuronal space database 2031, 3211 (step 107), it can be determined whether the shortest pathway exists between the two neurons or not by looking up the aforementioned shortest pathway tables (step 109). The step 107 and the step 109 are performed by the neuronal shortest pathway searching module 625.

When it is determined that the shortest pathway exists, the shortest pathway between the two neurons may be obtained (step 111). Otherwise, return to step 107 and reselect two neurons.

As described above, when the shortest pathway between any two neurons in the three-dimensional neuronal space database (also referred to as three-dimensional neuron image database) 2031, 3211 is to be searched, the step 109 may be utilized first to determine whether the shortest pathway exists or not. If yes, the shortest pathway between the two neurons may be found rapidly through the table formed from the predecessor matrixes.

In this embodiment, if the whole matrix cannot be loaded once due to the limitation of the system, the table may be split into several sub-pages and only the necessary sub-pages are loaded because the number of the nodes is about thirteen thousand and a 13000×13000 matrix includes a large amount of information. The loading and paging of sub-pages may be performed via the least recent used (LRU) method.

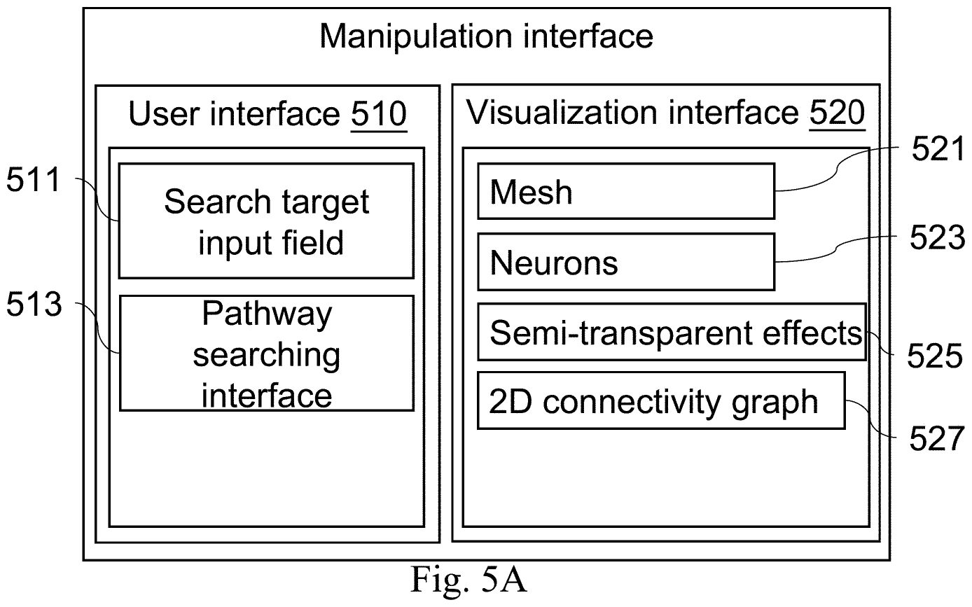

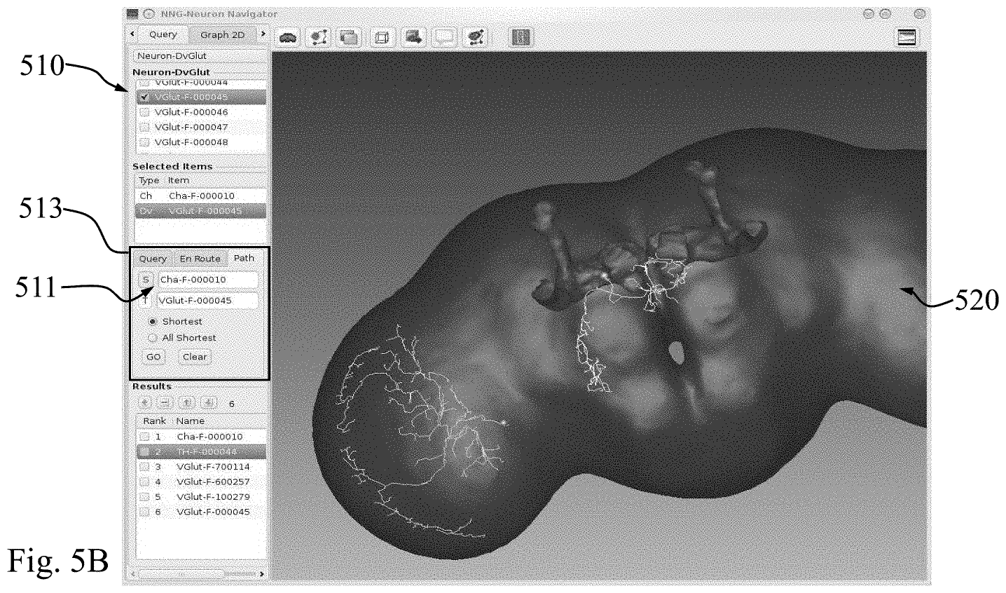

It should be noted that each step of the method for finding the shortest pathway between neurons disclosed by the present invention is performed by the system (processing device) 200 or the system 300. The manipulation interface 205, 319 may be performed as the structure disclosed in FIG. 5A. With reference to FIG. 5B, which is a diagram of the manipulation interface applied in the present invention in accordance with one embodiment, the manipulation interface 205, 319 may include two portions, which are the user interface 510 and the visualization interface 520 respectively.

The user interface 510 may include the following several functions but be not limited thereto: a pathway searching interface (Path Query) 513 and a search target input field (Query) 511, which enable the user to perform the manipulating action or the selecting action via the input device 209, 313. Furthermore, the visualization interface 520 may include the following several functions but be not limited thereto: mesh 521, neuron 523, semi-transparent effects 525- and two-dimensional connectivity graphs 527, which enable the user to recognize the neuronal images in a graphical way. Therefore, the user may manipulate the three-dimensional neuronal space database 2031, 3211 via the manipulation interface 205, 319 mentioned above, and after the user inputs the shortest pathway search commands, the computation may be performed by the processing device 201, 311.

Based on the above description, neurology researchers may establish the database by employing the neuronal images interested via the aforementioned method and can then find out the shortest pathways between every two neurons in the database easily and rapidly through the method described in the present invention, so as to facilitate the studies on neuronal functions and disease with persuasive anatomical descriptions.

It should be appreciated that the embodiments and the attached drawings are described and illustrated for purposes of illustration only, not for limiting, and that numerous alterations and modifications may be practiced by those skilled in the art without departing from the spirit and scope of the present invention. It is intended that all such modifications and alterations are included insofar as they come within the scope of the present invention as claimed or the equivalents thereof.

What is claimed is:

1. A system to find shortest pathways for neurons in neuronal linkage pathways, comprising:
a processing device;
a display coupled to said processing device;
a computer readable storing media coupled to said processing device, wherein a three dimensional neuronal apace database, shortest pathway tables and predecessor matrix tables are stored in said computer readable storing media and is executable by said processing device;
wherein said three dimensional neuronal apace database is processed by said processing device to define a unit voxel in a neuron image space as following steps of: determining whether each neuron in said three dimensional neuronal space database is connected to other neurons by said processing device, if said processing device determines any two neurons are located within same unit voxel, a connection exists between said two neurons, wherein said determining process is established by a connection matrix (CM) between said neurons from said three-dimensional neuronal space database;
wherein said shortest pathway tables are processed by said processing device as following steps of: calculating a shortest pathway table of all of said plurality of connected neurons through said connection matrix (CM) generated by a APSP (All-pairs Shortest Paths) algorithm by said processing device and storing said shortest pathway tables in said computer readable storing media; performing by said processing device to select said two neurons from a plurality of neurons and looking up said shortest pathway table to obtain a shortest pathway between said two neurons;
wherein said APSP algorithm is applied by said processing device to a computation of said connection matrixes, thereby generating two matrixes including a shortest path (SP) matrix and a predecessor matrix (Pred), wherein SP(i, j) refers to a distance of said short pathway from a node i to a node j; and
wherein said three-dimensional neuronal apace database is employed to find all possible neuron connections, said display showing said short pathway when said node i and said node j are determined.

2. The system of claim 1, wherein said neuron image space includes all registered neuronal images.

3. The system of claim 1, wherein said Pred(i, j)=nil (i.e. an empty set), said shortest pathway between said node i and said node j being Edge(i, j).

4. The system of claim 3, wherein said Pred(i, j)=k, wherein said shortest pathway from said node i to said node j passing a node k, wherein said node k is predecessor of said node j on said shortest pathway from said node i to said node j.

5. The system of claim 1, wherein said three-dimensional or higher dimensional neuronal neuronal space database is constructed from a plurality of said unit voxels.

6. The system of claim 1, wherein CM(i, j)=1 denotes that there is a connection between a neuron ni and a neuron nj; CM(i, j)=0 denotes that there is no connection between said neuron ni and said neuron nj.

7. The system of claim 1, wherein said shortest pathway matrix (SP) and said predecessor matrix (Pred) are generated in said All-pairs Shortest Paths algorithm.

8. The system of claim 7, wherein SP(i, j)=∞ denotes that there is no connection between a neuron ni and a neuron nj; Pred(i, j)=nil denotes that said shortest pathway between said node i and said node j is Edge(i, j)

9. The system of claim 8, wherein said Pred(i, j)=k denotes that said shortest pathway from said node i to said node j passes a node k, wherein said node k is a predecessor of said node j on said shortest pathway from said node i to said node j.

10. The system of claim 1, further comprising an augmented reality display device or a virtual reality display device coupled to said finding shortest pathways system.

11. A system to find shortest pathways for neurons in neuronal linkage pathways, comprising:
a processing device;
a display coupled to said processing device;
a computer readable storing media coupled to said processing device, wherein a three dimensional neuronal apace database, shortest pathway tables and predecessor matrix tables are stored in said computer readable storing media and is executable by said processing device;
wherein said three-dimensional neuronal apace database is processed by said processing device to define a unit voxel in a neuron image space, wherein a size of said unit voxel is set by said processing device as a threshold to determine whether terminals of two neurons are located within same said unit voxel; followed by determining whether each neuron in said three-dimensional neuronal space database is connected to other neurons by said processing device, if said processing device determines any two neurons are located within same unit voxel, a connection exists between said two neurons, wherein said determining process is established by a connection matrix (CM) between said neurons from said three dimensional neuronal space database;
wherein said shortest pathway tables are processed by said processing device as following steps of: calculating a shortest pathway table of all of said plurality of connected neurons through said connection matrix (CM) generated by a APSP (All-pairs Shortest Paths) algorithm by said processing device and storing said shortest pathway tables in said computer readable storing media; performing by said processing device to select said two neurons from a plurality of neurons and looking up said shortest pathway table to obtain a shortest pathway between said two neurons, wherein CM(i, j)=1 denotes that there is a connection between a neuron ni and a neuron nj; CM(i, j)=0 denotes that there is no connection between said neuron ni and said neuron nj;
wherein said APSP algorithm is applied by said processing device to a computation of said connection matrixes, thereby generating two matrixes including a shortest path (SP) matrix and a predecessor matrix (Pred), wherein SP(i, j) refers to a distance of said short pathway from a node i to a node j, if SP(i,j)=∞, it denotes that there is no pathway to connect said node i and said node j; and
wherein said three-dimensional neuronal apace database is employed to find all possible neuron connection and said display showing said short pathway when said node i and said node j are determined.

12. The system of claim 11, wherein said neuron image space includes all registered neuronal images.

13. The system of claim 11, wherein said Pred(i, j)=nil (i.e. an empty set), said shortest pathway between said node i and said node j being Edge(i, j).

14. The system of claim 13, wherein said Pred(i, j)=k denotes that said shortest pathway from said node i to said node j passes a node k.

15. The system of claim 14, wherein said node k is a predecessor of said node j on said shortest pathway from said node i to said node j.

16. The system of claim 11, wherein said three-dimensional or higher dimensional neuronal neuronal space database is constructed from a plurality of unit voxels.

17. The system of claim 11, further comprising a three-dimension neuronal space generating device coupled to said processing device.

18. The system of claim 11, further comprising a neurons connection determining device coupled to said processing device.

19. The system of claim 11, further comprising neuronal shortest pathway tables generating device coupled to said processing device.

20. The system of claim 11, further comprising a neuronal shortest pathway searching device coupled to said processing device.
